(19) United States
(12) Patent Application Publication
(10) Pub. No.: US 2025/0222216 A1
(43) Pub. Date: Jul. 10, 2025

(54) VENTILATION DEVICES, SYSTEMS, AND METHODS

(71) Applicant: ENGINEERING MEDICAL SOLUTIONS LLC, Madison, WI (US)

(72) Inventors: Nicholas Genthe, Madison, WI (US); Craig Hineline, Madison, WI (US); Zachary Genthe, Madison, WI (US)

(21) Appl. No.: 18/852,641

(22) PCT Filed: Mar. 30, 2023
(86) PCT No.: PCT/US2023/016864

§ 371 Date: Sep. 30, 2024

Related U.S. Application Data
(60) Provisional application No. 63/325,426, filed on Mar. 30, 2022.

(51) Int. Cl. A61M 16/00; A61M 16/06; A61M 16/08

(52) U.S. Cl. CPC A61M 16/0084; A61M 16/06; A61M 16/0816; A61M 2205/071; A61M 2205/18; A61M 2205/3523; A61M 2205/581; A61M 2209/088; A61M 2230/205

(57) ABSTRACT

Provided herein are devices, systems, and methods for delivering safe and effective amounts of air to subject in need thereof. In particular, devices, systems, and methods are provided that prevent over- or under-ventilation of subject.

The present application claims priority to U.S. Provisional Patent Application Ser. No. 63/325,426, filed Mar. 30, 2022, the disclosure of which is herein incorporated by reference in its entirety.

### FIELD

Provided herein are devices, systems, and methods for delivering safe and effective amounts of air to subject in need thereof. In particular, devices, systems, and methods are provided that prevent over-or under-ventilation of subject.

### BACKGROUND

Existing manual resuscitators have grave inadequacies. A number of these problems are described well in U.S. Pat. No. 7,121,278, which are highlighted below. One of the first priorities in resuscitation is to provide artificial ventilation to a patient. This begins with establishing an airway, which is the process of providing an open passage for air to travel through the patient's mouth, throat, and trachea (or windpipe) to the lungs. This can initially be achieved by properly positioning the patient's head and neck. Once an airway is established, one next provides artificial ventilation by forcing air into the patient's mouth, through the trachea, and into the lungs. When utilizing the mouth-to-mouth method of ventilation, no ventilatory device is used. This is the form of artificial breathing taught by the American Heart Association (AHA) and the European Resuscitation Council (ERC) as part of Basic Life Support (BLS) courses on cardiopulmonary resuscitation (CPR).

However, healthcare professionals, both in-hospital (physicians, respiratory therapists, nurses, etc.) and pre-hospital (emergency medical technicians, paramedics, first responders, etc.) most often use various medical devices to provide artificial ventilation. These can include pocket masks (through which the rescuer manually blows to inflate the patient's lungs), demand valves (a mechanical device which inflates the patient's lungs with compressed oxygen when a button is manually pressed on the face mask), and automatic transport ventilators (fully automatic mechanical ventilators which deliver sequential breaths to the patient). The device which is most frequently employed is the manual resuscitator.

Also known as a “bag-valve-mask” (BVM), the manual resuscitator is a balloon-type device. Essentially a hand-powered air pump, the device consists of a squeezable, pliable self-inflating bag (or fluid chamber) which, when squeezed by the operator, displaces air from the bag and out a port to which a face mask can be connected. More specifically, the technique to use a manual resuscitator often comprises utilizing one hand to perform the combined task of maintaining proper positioning of the head (to maintain the airway), while applying pressure on the mask to form a seal between the patient's face and the mask. At the same time, the operator uses the free hand to squeeze the fluid chamber and thus displace air into the patient's lungs under positive pressure. When the fluid chamber is released, the patient passively exhales while the fluid chamber returns to its natural, inflated state in preparation for the next breath.

While manual resuscitators can, in theory, be used safely and effectively, in practice, they are frequently misused. One problem identified is a general inability for single rescuers to simultaneously use one hand to maintain the face mask seal while using the other hand to squeeze the chamber to generate a breath of sufficient volume (called the tidal volume). One reason for these low volumes is it is difficult to maintain an airtight seal between the face mask and the patient's face with one hand, resulting in frequent loss of a significant portion of the tidal volume to leakage. Another problem contributing to small tidal volumes is the randomly pliable nature of the skin of the fluid chamber of the device. When squeezed, areas not in direct contact with the rescuer's hand bulge out, reducing the efficiency of the manual compressing action which constitutes operation of the device. Studies have demonstrated that these deficiencies are not related to the level of training of the rescuer—paramedics, nurses, and physicians operating the device were all found to be unable to consistently provide ventilation at recommended levels (Khoury et al., BioMed Research International, vol. 2016, Article ID 4521767, 2016). While it may seem intuitive that this problem can be overcome simply by providing more frequent breaths, this strategy can result in further decreased ventilation to the patient.

This apparent paradox, where increasing ventilatory rate may lead to decreased overall ventilation of the lungs, is related to a physiologic principle known as anatomic deadspace. The actual exchange of gases in the lungs, called respiration, occurs in tiny air sacks that are surrounded by a web of blood capillaries. These sacks, called alveoli, is where the blood receives oxygen from the air inhaled in exchange for carbon dioxide waste, which is exhaled. In every individual, a significant portion of a given breath remains in the mouth, throat, trachea, and the various distal airways in the lungs, which are collectively referred to as deadspace. Residual air occupying deadspace at the end of inhalation never actually reach the alveoli and therefore do not contribute to gas exchange. Accordingly, since deadspace is an anatomic constant unaffected by the size of the breath administered, when small breaths are given, deadspace negates 25-35% the of total tidal volume delivered, whereas if large breaths are given, deadspace consumes only 10-20% of each tidal volume. Consequently, one ventilating rapidly but with small tidal volumes is likely to deliver less effective ventilation than one would by utilizing a larger tidal volume at a slower rate.

This paradox has significant clinical implications. Frequently during resuscitation, certain blood tests are performed that measure the amount of oxygen and carbon dioxide in the blood. When such examinations reveal decreased oxygen levels or, more importantly, elevated amounts of carbon dioxide in the blood, the individual ventilating is usually prompted to increase their efforts. The natural response would be to increase the ventilatory rate, however, higher ventilatory rates have been associated with increased operator hand fatigue and inattentiveness. Consequently, tidal volumes have been observed to decrease as ventilatory rates increase. Therefore, despite increased ventilatory rate (and operator impression they are providing improved ventilation), overall ventilatory effectiveness may decrease, because as tidal volume decreases, anatomic deadspace represents increasing proportions of each breath, which can provide a greater negative affect on alveolar ventilation than the positive effect of a higher rate.

This concept is not universally recognized among health care providers. As a result, many continue to inappropriately regard the effectiveness of the manual resuscitator as rate-dependent rather than volume-dependent.

Because of this inability of single rescuers to generate adequate volumes, authoritative agencies recommended implementation of a two-person technique to utilize manual resuscitators—one-person to maintain a face mask seal with two hands, while the other rescuer squeezes the fluid chamber using two hands. Clinical studies performed thereafter sought to document delivery of higher tidal volumes consistent with resuscitation standards.

While increased volumes are produced by the two-person technique (Beitler et al. (2017). American Journal of Respiratory and Critical Care Medicine, 195(9), 1198-1206), clinical studies also identified significant hazards associated with the two-person technique. To compensate for the aforementioned bulging-out phenomenon during the one-handed technique, resuscitator manufacturers make fluid chambers disproportionally large. Thus, when two hands are used to provide a breath, improved surface-area contact between the hands and the fluid chamber decrease the extent of outward bulging, resulting in the generation of excessive volumes, air flow rates, and airway pressures.

Generation of excessive volumes, pressures, and flow rates has been shown to cause significant hazards to the patient. One study assessed the distribution of gas between the lungs and stomach in patients ventilated with manual resuscitators (Barnes, T. A., & Ward, J. J. (2018). Respiratory Care, 63(5), 635-636). Even with the one-person technique, air flow rates and airway pressures were excessive enough to cause air to preferentially enter the stomach, and at times, flow to the stomach was greater than the amount received by the lungs. Inflation of the stomach with air (called gastric insufflation) markedly increases the risk of patient vomiting, potentially resulting in stomach contents entering the lungs (a major complication). In addition to the risk of gastric insufflation, over-ventilation poses a specific risk of reducing venous return during cardiopulmonary resuscitation (CPR). Chest compression and manual ventilation are the mainstay of CRP. In the scenario when manual ventilation with a BVM is occurring simultaneously with chest compression, over ventilation has been shown to be associated with lower rates of return of spontaneous circulation (ROSC). This is theorized to be because as a breath is delivered it increases the intrathoracic pressure within the chest, which transiently decreases the rate of venous return to the heart as well as effective forward flow during that compression (Aufderheide et al. (2004). Circulation, 109(16), 1960-1965). In fact, the danger and incidence of gastric distention and reduced venous return associated with the use of prior art manual resuscitators has recently been determined to be great enough to recommend utilization of child-size versions of the devices on adult patients, since the smaller size of the child device provides a safeguard against generation of excessive volumes, pressures, and flow rates which may lead to a decreased incidence of these complications. Accordingly, some resuscitation authorities recommended that, in lieu of a truly safe and effective ventilatory adjunct, when ventilating an adult with existing devices, it is preferable to compromise ventilatory effectiveness in order to achieve greater security from complications associated with adult-versions of the device.

In patients who are successfully resuscitated, high inflation pressures have been shown to be a contributing factor to the development of the Acute Respiratory Distress Syndrome (ARDS), the treatment of which requires sustained mechanical ventilation and intensive-care hospitalization over several weeks or months. Treatment of ARDS is extensive, extremely costly, and frequently unsuccessful. Excessive ventilatory volumes and pressures can also cause acute life-threatening lung injury through actual disruption of lung tissue. In addition to pneumothorax (collapsed lung), there have been several reports of cases where high inflation pressures generated by manual resuscitators have caused air to directly enter the bloodstream (called an air embolism), a complication which is almost invariably fatal. Even in patients with a perfusing rhythm, high airway pressures are known to decrease blood pressure, cardiac output, and oxygen delivery significantly by causing the lungs, inflated with high pressures, to compress the heart and the large blood vessels in the chest.

Another deficiency of existing devices is related to high variability of tidal volumes generated by the device. Small changes in hand position on the fluid chamber are exaggerated by the bulging-out effect, causing disproportionate changes in tidal volumes. This attribute contributes to the inability of the existing devices to provide consistent and predictable ventilation to the patient, regardless of operator technique. This has been postulated to interfere with the ability to interpret certain blood tests which assess the effectiveness of ventilation and that are fundamental measures of ventilation effectiveness.

Another disadvantage associated with breath-to-breath inconsistency is an inability to detect certain life-threatening conditions that are associated with increasing lung resistance. The presence of a life-threatening lung injury (e.g., tension pneumothorax) may be detected early by noticing a requirement for progressively increasing ventilatory pressures to achieve delivery of a specific tidal volume. With marked breath-to-breath variation associated with existing devices, this condition is likely to be apparent only after advanced progression of the injury begins to contribute to circulatory collapse and appearance of other ominous physical findings. Late identification of such underlying injuries further jeopardizes the patient and complicates treatment.

There are also significant and severe health ramifications to delivering breaths too slow or too fast. Health professionals focus on “minute ventilation,” which is defined as the tidal volume delivered multiplied by the number of deliveries per minute. For example, 600 mL tidal volume times 10 breaths per minute results in a minute ventilation of 6,000 mL/min. Delivering breaths too slowly can result in a significantly lower minute ventilation, which starves the patient of oxygen and allows carbon dioxide to build in the body. Delivering breaths too frequently can purge CO₂ from the body too quickly, causing pH levels in the blood to rise and leading to cerebral vasoconstriction and reduced blood flow to the brain. The target ventilation rate taught in by the American Heart Association on cardiopulmonary resuscitation (CPR) is one breath every six seconds (10 breaths per minute). Studies have found that in real situations, that are often extremely intense and stressful, the average delivery rate of artificial breaths is closer to one every 3.2 seconds (nearly 19 breaths per minute), even when the operator is professionally trained (Culbreth, et al. (2021). Heart & Lung, 50(3), 471-475).

Accordingly, existing devices, while simple and inexpensive in design and operation, have multiple barriers to the consistent delivery of safe and effective artificial ventilation. Its use in unintubated patients either results in inadequate ventilation and hypercarbia with the one-person technique (or if a child-size device is employed), or the unacceptable risk of gastric insufflation and aspiration of vomitus associated with the two-person technique with the full-sized adult device. When used on intubated patients, employment of a one-handed technique contributes to hypoventilation and hypercarbia, the latter of which has been proven to have several potent effects on the heart which directly contribute to increased mortality from cardiac arrest. When two-handed operation is used on intubated patients, the lack of an ability to guard against the generation of excessive airway pressures and volumes has been demonstrated to result in circulatory depression secondary to decreased venous return as well as a significant incidence of lung injury, both of which results in further complications, increased hospitalization, and/or death. Furthermore, breath-to-breath variability associated with the existing devices results in unpredictable and inconsistent ventilation (affecting the ability to interpret certain blood tests), and decreases or inhibits the sensitivity for one to detect progressively increasing pulmonary resistance to ventilation, which can be indicative of endotracheal tube displacement or the presence of underlying life-threatening intrathoracic injury. Lastly, the rate that rescue breaths are administered in the field vastly differs from the prescribed rates, and therefore the actual minute ventilation vastly differs from the prescribed minute ventilation, which can cause pH levels in the blood to rise and lead to cerebral vasoconstriction and reduced blood flow to the brain (Spaite et al. JAMA Surgery, 154(7)).

Thus, improved devices and methods of using them are needed.

### SUMMARY

As described in the Background section above, there are significant problems with existing devices used for manual resuscitation. These problems have been known for a long time and while a number of solutions have been attempted, none have gained traction in the marketplace or solved the problems sufficiently. Provided herein are devices, systems, and methods that solved these problems.

In accordance with the present invention an improved design for a manual resuscitator is provided comprising a hinged device with mechanical stopping features and/or timing features attached to, attachable to, or incorporated into, the squeezable BVM fluid chamber. The device is pre-calibrated or field adjustable to provide an upper limit on the volume squeeze of the bag, reducing issues with over-delivery of air volume to the patient. Under-delivery of air volume to the patient is prevented so long as the operator squeezes until the mechanical stop and/or tactile feedback is felt. The device also provides a uniform squeeze of the bag, eliminating outward bulging of the bag during operation. The timing features (e.g., timer with indicating light, vibrating indication, or other alarm mechanism) guide the rescuer to the prescribed ventilation rate recommended by the American Heart Association (AHA) and European Resuscitation Council (ERC). As shown by the data presented herein, these features provide a significant improvement in air flow management.

In some embodiments, provided herein are devices, systems, and methods employing a hinged mechanical device that enables uniform and predictable compression of a fluid chamber without regard to hand placement, size of the operator's hands, or use of one or two hands for operation.

In some embodiments, provided herein are device, systems, and methods that consistently provide full, effective, and uniform tidal volumes and minute ventilations without regard to technique, in alignment with the AHA and ERC recommendations.

In some embodiments, provided herein are devices, systems, and methods that provide safeguards to prevent patient harm caused by the delivery of excessive tidal volumes, airway pressures, and flow rates.

In some embodiments, provided herein are devices, systems, and methods that enable assertive delivery of large tidal volumes without jeopardizing patient safety, thus providing safer, more effective ventilation.

In some embodiments, provided herein are devices, systems, and methods that provide consistent volumes with each breath, increasing the uniformity of airway pressures sensed by the operator and accordingly the ability to detect progressively increasing airway resistance, which also increases the clinical applicability of certain monitoring tests.

In some embodiments, provided herein are devices, systems, and methods that provide a light, vibration, sound, and/or other signals to the rescuer that assist in keeping the prescribed cadence (delivery rate) of rescue breaths.

In some embodiments, provided herein are devices, systems, and methods that limit excessive tidal volumes able to be administered to patients, thus decreasing the incidence and significance of gastric insufflation and risk to the unprotected airway.

In some embodiments, provided herein are devices, systems, and methods that provide the ability to accurately and consistently provide the prescribed minute ventilation, via a device that guides both the rate and volume of rescue breaths.

For example, in some embodiments, provided herein is a resuscitation device, comprising: a) an inflatable bag; b) a mouthpiece in fluid communication with the inflatable bag; and c) a volume-regulating hinged device comprising: i) first and second panels in contact with the bag; and ii) a hinge connecting the first and second panels; wherein the hinge is configured to limit movement of the first and second panels towards each other to a pre-defined distance so as to restrict a volume of air delivered from the bag to the mouthpiece when the hinged device is moved to a closed position. In some embodiments, three or more panels are employed. In some embodiments, two or more hinges are employed.

In some embodiments, provided herein is a system comprising: a) a resuscitation device comprising an inflatable bag and a mouthpiece in fluid communication with the inflatable bag; and b) a volume-regulating hinged device comprising: i) first and second panels configured to contact an exterior surface of the bag; and ii) a hinge connecting the first and second panels; wherein the hinge is configured to limit movement of said first and second panels towards each other to a pre-defined distance so as to restrict a volume of air delivered from the bag to the mouthpiece when the hinged device is moved to a closed position when in contact with the exterior surface of the bag. In some embodiments, three or more panels are employed. In some embodiments, two or more hinges are employed.

In some embodiments, the first and/or second panels are arcuate in shape.

In some embodiments, the hinge comprises a stop that prevents movement of the panels beyond the pre-defined distance. In some embodiments, contact of panel one and panel two creates a stop that prevents movement of the panels beyond the pre-defined distance. In some embodiments, the contact at the stop generates an audible, visual, and/or tactile feedback mechanism.

In some embodiments, the first and second panels physically contact each other (e.g., at their junctures closest to the hinge) at the pre-defined distance, preventing further closure of the hinged device.

In some embodiments, the systems or devices further comprise an alarm. In some embodiments, the alarm is configured to alert a user of an optimal time for closing (e.g., manually closing) the hinge. In some embodiments, the alarm comprises one or more of a tactile response, a light, or a sound. In some embodiments, the optimal time is a preset constant value. In some embodiments, the preset constant value is approximately 6 seconds. In some embodiments, the optimal time is adjustable based on airflow feedback. In some embodiments, the airflow feedback comprises biofeedback from a ventilated subject. In some embodiments, the biofeedback comprises blood oxygen level.

In some embodiments, the hinged device comprises a strap sized to fit a user hand. In some embodiments, the strap is positioned to hold the user hand to the hinged device when user fingers contact the first panel and user palm contacts the second panel.

In some embodiments, the hinged device comprises an attachment component (e.g., adhesive, hook, hook and loop fasteners (e.g., VELCRO fasteners), latch, snap, magnet, etc.) configured to fix the hinged device to the bag.

In some embodiments, the hinged device comprises an attachment component configured to fix the hinged device to a rigid component on the top or bottom of the BVM (see e.g., FIG. 1).

In some embodiments, a BVM is manufactured with an integrated hinged device. For example, in some embodiments, the hinged device is fabricated into and continuous with the surface of a BVM.

Also provided herein are methods of resuscitating a subject, comprising: a) contacting a device or system as described above or elsewhere herein to a mouth and/or nose of a subject; and b) closing the hinged device to compress the bag. In some embodiments, the step of closing the hinged device is timed in response to an alarm produced by the system or device.

Also provided herein are stand-alone devices for regulating air flow with a bag-type resuscitation system, comprising one or more or each of: a) a first arcuate panel having an inner surface configured to mate to an outer surface of a bag of a bag-type resuscitation system; b) a second arcuate panel having an inner surface configured to mate to the outer surface of the bag; c) a hinge connecting the first and second arcuate panels and allowing the first and second arcuate panels to move towards one another; d) a stop mechanism configured to prevent movement of the first and second arcuate panels towards one another beyond a pre-determined threshold distance; e) a strap mounted on an outer surface of the first arcuate panel or the second arcuate panel and shaped to receive a human hand; and f) an alarm configured to provide sensory feedback to a user at predetermined time intervals.

In some embodiments, a component is provided to measure, directly or indirectly, radial movement of the hinge, such that volume at any given time is determined. In some embodiments, a sensor is used to provide a relative angle measurement of the hinged device.

In some embodiments, the system or any component thereof (e.g., the hinged device) comprises a communication component to allow information to be transferred from the system to a remote device (e.g., smart phone, computer, data cloud, etc.). In some embodiments, the communication component provides BLUETOOTH or WIFI wireless technology for communication to an external system or device.

In some embodiments, one or more rechargeable or disposable batteries is provided to power any electronic components of the system (e.g., alarms, sensors, data collection components, communication components).

### DESCRIPTION OF FIGURES

FIG. 1 shows an exemplary volume regulator (4) attached to a bag-valve-mask resuscitation device (1).

FIG. 2 shows an exploded view of an exemplary volume regulating hinged device (4).

FIG. 3 shows an alternative design of an exemplary volume regulating hinged device (4).

FIG. 4. shows an alternative exemplary volume regulator (4) in exploded view.

FIG. 5 shows an exemplary configuration for a single-user implementation of a system where the bag (2) is in an open (uncompressed) position.

FIG. 6. shows an exemplary configuration for a single-user implementation of a system where the bag (2) is in a closed (compressed) position.

FIG. 7. shows an exemplary configuration for a two-user implementation of a system.

FIG. 8. provides test data obtained from an experimental trial using a system.

FIG. 9. provides a graphical representation of test data obtained from an experimental trial using a system.

### FIGURE KEY

1. Resuscitation Device
2. Inflatable bag
3. Patient breathing interface
4. Volume regulated hinged device
5. First panel
6. Second panel
7. Hinge
8. Fixed or adjustable strap
9. Alarm
10. On/off switch
11. Strap
12. Bag attachment component
13. Hinge pin
14. Screw
15. Electronics cover
16. Electronics
17. Slot for strap
18. Features to enhance user's grip

### DETAILED DESCRIPTION

Provided herein are devices, systems, and methods for delivering safe and effective amounts of air to subject in need thereof. In particular, devices, systems, and methods are provided that prevent over-or under-ventilation of subject.

Certain exemplary embodiments of the systems, devices, and methods are described in detail in this section. It should be understood that the invention is not limited to these specific illustrative examples.

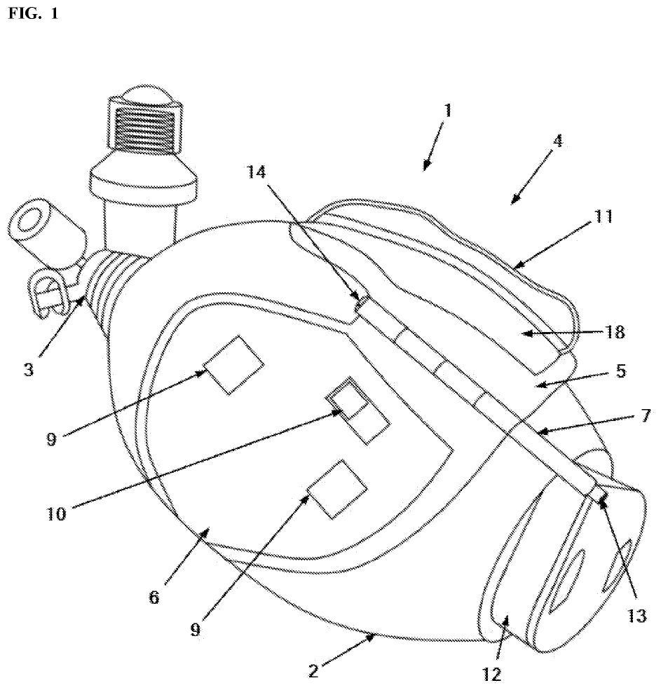

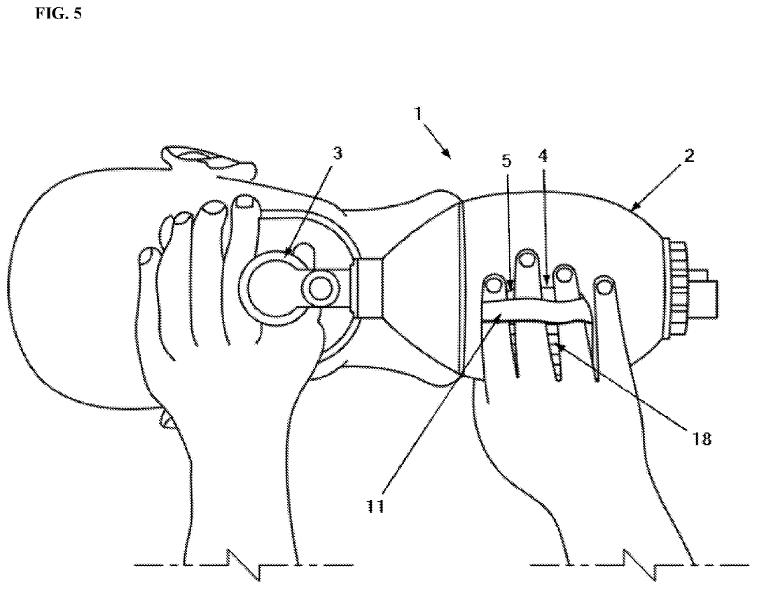

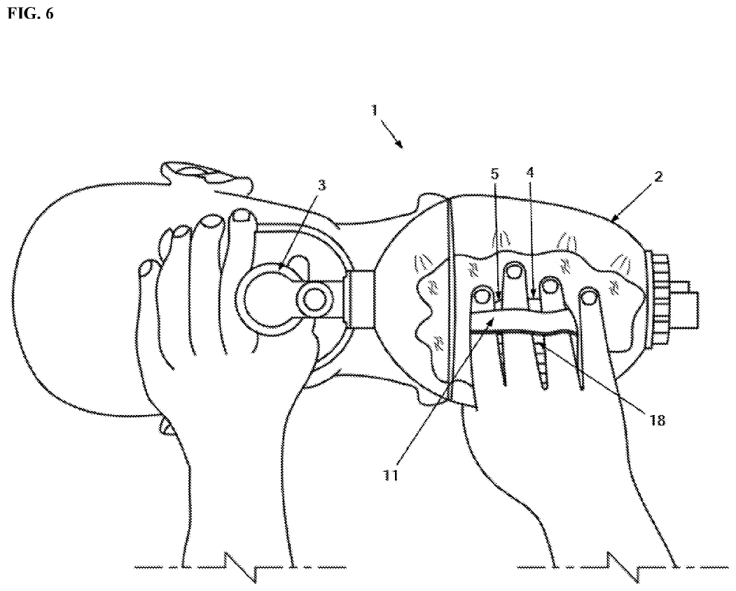

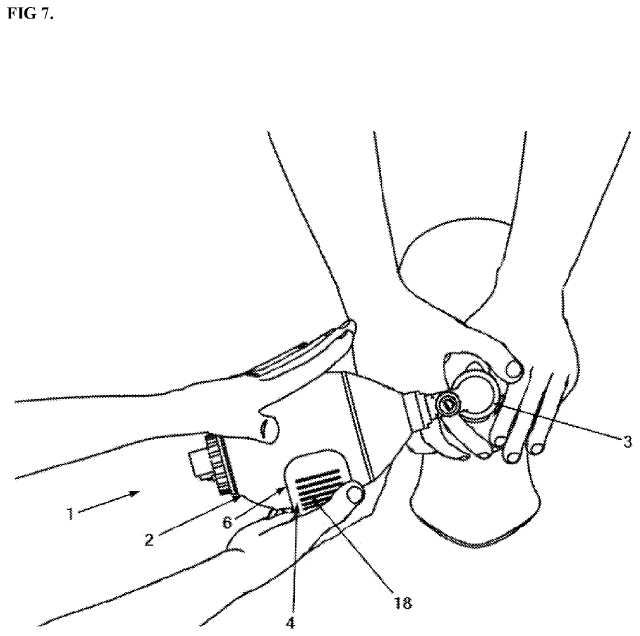

FIG. 1, FIG. 5, FIG. 6, and FIG. 7 depict exemplary systems of the present invention. FIG. 1 shows an exemplary embodiment of a resuscitation device (1) comprising of a volume regulated hinged device (4), an inflatable bag (2) and a patient breathing interface (3). FIG. 5 shows a system with use by one person. FIG. 6 shows the same as FIG. 5, but with the inflatable bag (2) in a compressed state. FIG. 7 shows how the system would typically be used with 2 persons.

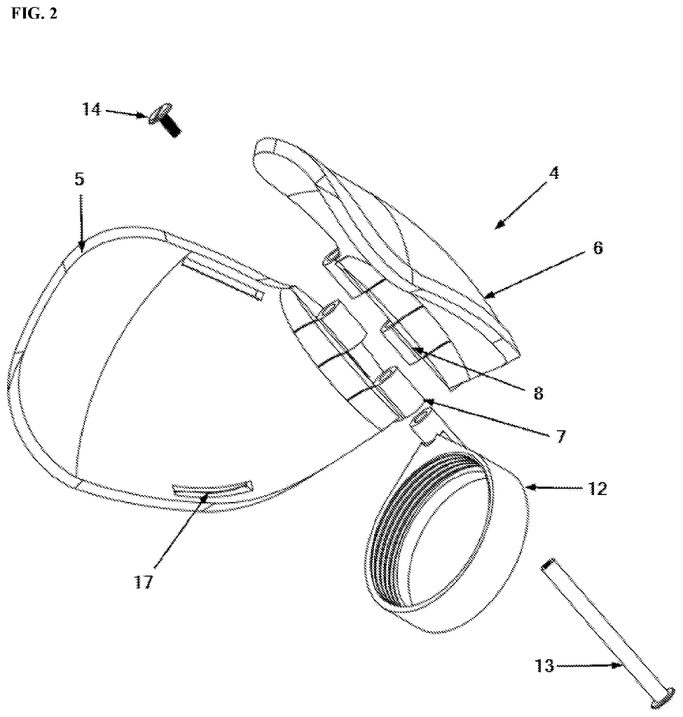

FIG. 2 depicts an exemplary design of a volume regulated hinged device (4) in an exploded view.

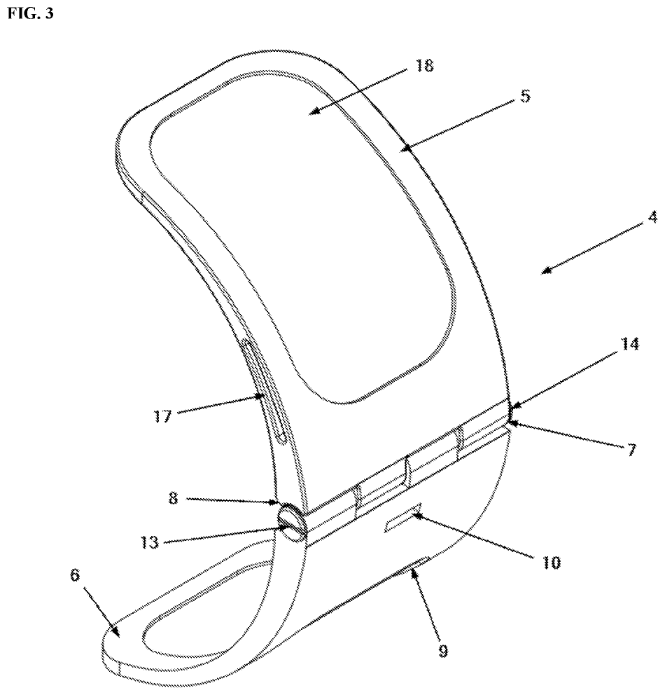

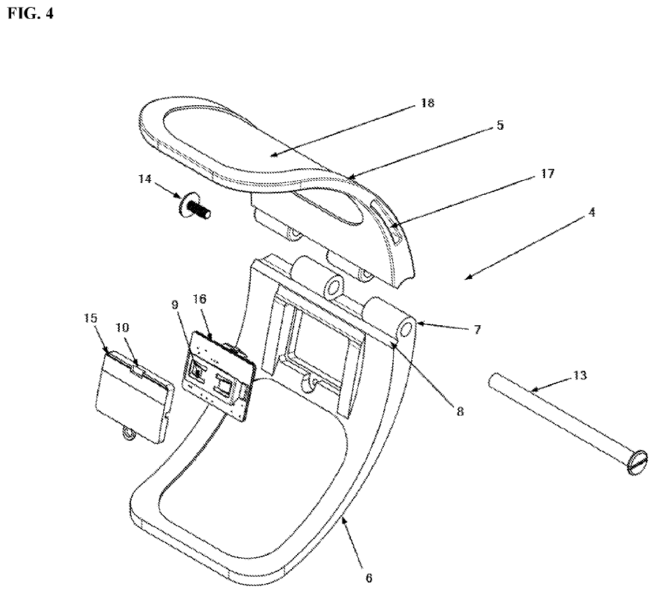

FIG. 3 and FIG. 4 depict an alternative exemplary design of a volume regulated hinged device (4) in different views, exploded and non-exploded.

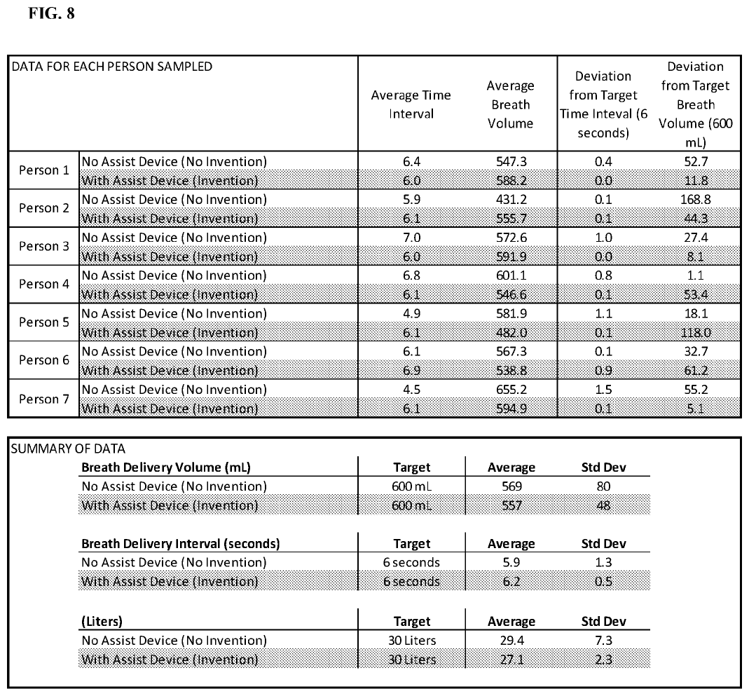

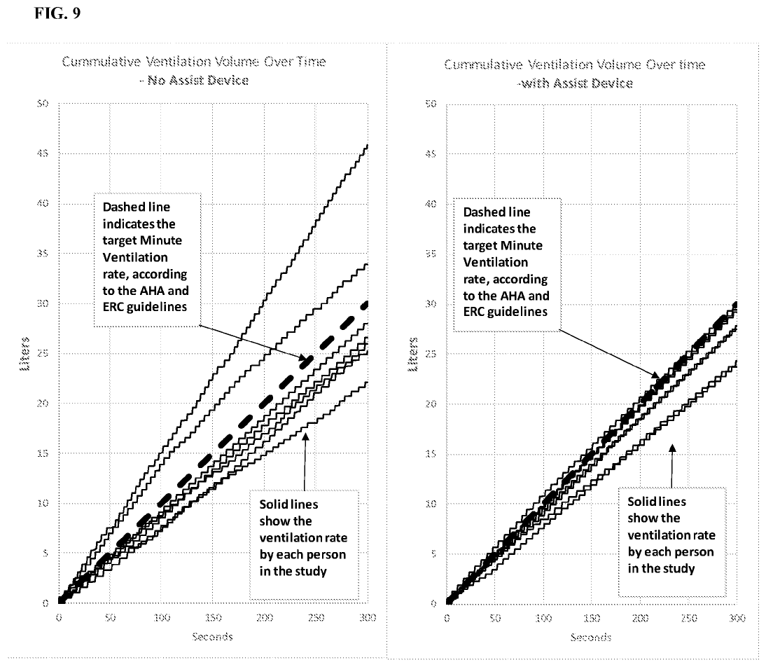

It can be seen from the data in FIG. 8 and FIG. 9 and described in the Example section below that the benefits provided by the inventive design provide several functional and operational advantages over the prior art. As mentioned previously, one of the disadvantages of the prior art is the highly unpredictable amount of volume generated by the device in each breath is, making it difficult for the user to anticipate the degree of ventilatory support provided to the patient. In contrast, the volume restricting mechanism of the present invention provides a new ability to achieve prescribed tidal volume to be delivered with each breath. Additionally, even if hand placement on the inventive device is off-center, such as toward one end rather than in the middle, the device will still compress downward in a uniform movement. The ability to accurately deliver volume to the patient in accordance with American Heart Association and the European Resuscitation Council will greatly contribute to safety by avoiding patient exposure to excessive volumes that can contribute to lung injury.

A secondary benefit demonstrated by the experimental testing was the visual simplicity and intuitive nature when used with the standard BVM resuscitators that are most often employed during emergent life-saving efforts. Clinicians are often reluctant to worsen the complexity of their tasks by experimenting with new, unfamiliar devices substantially different from those presently in use, and in particular any pertaining to ventilation which is so crucial to patient survival. The present invention effectively incorporates the desired functional attributes and improvements in clinical capability while substantially preserving utilization techniques in present use with the current bag valve mask standard of care. In some embodiments, the present invention is easily removed from the BVM at any moment, which gives further comfort to the healthcare provider. Little additional training is required to use the systems, devices, and methods of the invention. Furthermore, the device provides a cognitive offload for the provider. The provider no longer needs to count the seconds between breaths in their head, for example, but instead is alerted by the present invention that it is time to administer the next breath.

In some embodiments, the systems and devices comprise a combination of one or more of a volume restrictor (4), a timer and timing indicators (9), for use in conjunction with, or as an integrated part of, a manual resuscitation device (1), commonly known as a Bag Valve Mask (BVM), which comprises of a patient breathing interface (3) and an inflatable bag (2).

FIG. 1 through FIG. 7 show several views of an exemplary volume restrictor in the form of a hinged device (4). The volume restrictor (4) consistently provides predictable and uniform generation of gas flow through the inflatable bag (2) i.e. BVM for ventilation without regard to one or two-handed technique, hand placement, hand strength, or hand size. Where the volume restrictor (4) is provided as a separate component, the volume restrictor may comprise an attachment component (12) to attach to the resuscitation device (1). Any mechanism that can temporarily or permanently attach or associate the volume restrictor (4) to the resuscitation device (1) may be employed as an attachment component (12), including, but not limited to, adhesives (e.g., two-sided adhesive tape), hook and loop fasteners, snaps, hooks, magnets, and the like. In some embodiments, the attachment is readily reversible, allowing for easy detachment.

In some embodiments, the systems and devices comprise a stopping mechanism (8) that restricts the angle in which the first panel (5) and second panel (6) of a hinged device (4) can pivot towards one another. The hinged device typically would comprise of a hinge pin (13), hinge (7), and screw (14). In some embodiments, the systems and devices comprise a hinge (7) separating opposite panels of a hinged device volume restrictor (4).

In some embodiments, a component (e.g., a strap) (11) is provided on the volume restrictor (4) to assist with maintaining a user's hand in correct operational position on the volume restrictor (4). For example, a strap (11) may be provided on a surface of one or more sides of the volume restrictor (4) that is opposite the side that makes contact with the bag (2). The device may have a slot (17) designed specifically to hold the strap (11). In some embodiments, there are features to enhance a user's grip (18), such as ridges, grooves, and/or rubberized materials such as grip tapes, and the like.

In some embodiments, an alarm (9) is provided. The alarm (e.g., timer) may be mechanical, but preferably electrical, e.g., the electronics (16) and may be encased in the hinged device (4) by an electronics cover (15). In some embodiments, the alarm (9) is provided in a computer chip and calculates a time sequence corresponding to a desired actuation rate for the volume restrictor (4). In some embodiments, an alarm is provided that provides a sensory indication to the user of the time sequence. The alarm (9) may comprise light (e.g., from LED lights), sound, tactile feedback (e.g., vibration) from a vibration element, or other sensory response. In some embodiments, the alarm (9) comprises an electronic communication component that allows an external device (e.g., earbuds, phone, headset, etc.) to generate the alarm that notifies the user when to actuate the bag (2) using the volume restrictor (4). In some embodiments, an on/off switch (10) is provided that activates and deactivates the timer and/or alarm (9) components (e.g., to save battery life). In some embodiments, a switch, dial, or other user control interface is provided that allows a user to adjust the timer settings. In some embodiments, the electronics (16) may further comprise a communication component. The communication component may provide wireless or wired transfer of information to or from the device or system. For example, in some embodiment information is transmitted wirelessly to an external system or device (e.g., phone, computer, server, etc.). In some embodiments, information (e.g., settings information) is received by the device from an external system or device.

The volume restrictor (4) may be any desired size. Sizes may be selected to account for different inflatable bag (2) sizes (e.g., adult versus pediatric bags) or user hand sizes. Alternative sizes may also be selected if the system is used on non-human (e.g., veterinary) subjects, where smaller and larger bags may be employed.

In some embodiments, the device or system is designed for single use and is disposable. In some embodiments, one or more components are designed for multiple uses. In such embodiments, materials may be selected that allow for sterilization (e.g., autoclaving).

### EXAMPLE

A brief simulation study was conducted to validate the effectiveness of the invention.

Seven test subjects were gathered to perform rescue breathing on a CPR dummy. The people consisted of two medical doctors, a nurse, and 4 non-qualified healthcare professionals (non-QHP) persons. The ratio was 4 males and 3 females. Rescue breathing (manual resuscitation) was performed with a standard bag-valve-mask. A flowmeter designed specifically for analysis of ventilation between the bag and the mouth seal connector, a Sensirion Mass Flowmeter model SFM3200, was employed.

Each person performed rescue breathing on the CPR dummy twice, for 5-minute durations each time. The first time was without a system of the invention. The second 5-minute duration of rescue breathing was performed with a system of the invention.

The flowmeter collected flowrates every 1/100th of a second. The flowrates were integrated (totalized) to determine volumes delivered for each artificial breath that was given.

The results are presented in FIG. 8 and FIG. 9. The results demonstrate that a system of the invention improves both the accuracy and the precision of breaths delivered-for both the time interval of breaths as well as the breath volumes delivered. The use of the system of the invention also prevented any substantial over-ventilation.

We claim:

1. A resuscitation device, comprising:
a) an inflatable bag;
b) a patient breathing interface in fluid communication with the inflatable bag; and
c) a volume-regulating hinged device comprising:
i) first and second panels in contact with said bag; and
ii) a hinge connecting the first and second panels;
wherein said hinge is configured to limit movement of said first and second panels towards each other to a pre-defined distance so as to restrict a volume of air delivered from the bag to the mouthpiece when the hinged device is moved to a closed position.

2. A system comprising:
a) a resuscitation device comprising an inflatable bag and a patient breathing interface in fluid communication with the inflatable bag; and
b) a volume-regulating hinged device comprising:
i) first and second panels configured to contact an exterior surface of said bag; and
ii) a hinge connecting the first and second panels;
wherein said hinge is configured to limit movement of said first and second panels towards each other to a pre-defined distance so as to restrict a volume of air delivered from the bag to the mouthpiece when the hinged device is moved to a closed position when in contact with the exterior surface of said bag.

3. The system or device of claim 1 or 2, wherein said patient breathing interface is a mouthpiece.

4. The system or device of claim 1 or 2, wherein said patient breathing interface is a tube.

5. The system or device of claim 1 or 2, wherein said device comprises three or more panels and two or more hinges.

6. The system or device of claim 1 or 2, wherein said volume-regulating hinged device is integral with said inflatable bag.

7. The system or device of claim 1 or 2, wherein said volume-regulating hinged device is detachably removable from said inflatable bag.

8. The system or device of claim 1 or 2, wherein the first and second panels are arcuate.

9. The system or device of claim 1 or 2, wherein said hinge comprises a fixed or adjustable stop that prevents movement of said panels beyond said pre-defined distance.

10. The system or device of claim 1 or 2, wherein said first and second panels physically contact each other at said pre-defined distance, preventing further closure of said hinged device.

11. The system or device of claim 1 or 2, further comprising an alarm.

12. The system or device of claim 11, wherein said alarm is configured to alert a user of an optimal time for closing said hinge.

13. The system or device of claim 11, wherein said alarm comprises one or more of a tactile response, a light, or a sound.

14. The system or device of claim 11, wherein said alarm comprises an On/Off switch.

15. The system or device of claim 12, wherein said optimal time is a preset constant value.

16. The system or device of claim 15, wherein said preset constant value is approximately 6 seconds.

17. The system or device of claim 12, wherein said optimal time is adjustable based on airflow feedback.

18. The system or device of claim 17, wherein said airflow feedback comprises biofeedback from a ventilated subject.

19. The system or device of claim 18, wherein said biofeedback comprises blood oxygen level.

20. The system or device of claim 1 or 2, wherein said hinged device comprises a strap sized to fit a user hand.

21. The system or device of claim 20, wherein said strap is positioned to hold the user hand to the hinged device when user fingers contact the first panel and user palm contacts the second panel.

22. The system or device of claim 1 or 2, wherein said hinged device comprises an attachment component configured to fix the hinged device to the bag.

23. The system or device of claim 1 or 2, further comprising a communication component that transmits and/or receives wireless information.

24. A method of resuscitating a subject, comprising: a) contacting a device or system of any of claims 1-23 to a mouth, nose, or airway of a subject; and b) closing said hinged device to compress said bag.

25. The method of claim 24, wherein the step of closing said hinged device is timed in response to an alarm produced by said system or device.

26. A device for regulating air flow with a bag-type resuscitation system, comprising:
a) a first arcuate panel having an inner surface configured to mate to an outer surface of a bag of a bag-type resuscitation system;
b) a second arcuate panel having an inner surface configured to mate to the outer surface of the bag;
c) a hinge connecting the first and second arcuate panels and allowing said first and second arcuate panels to move towards one another;
d) a stop mechanism configured to prevent movement of said first and second arcuate panels towards one another beyond a pre-determined threshold distance;
e) a strap mounted on an outer surface of said first arcuate panel or said second arcuate panel and shaped to receive a human hand; and
f) an alarm configured to provide sensory feedback to a user a predetermined time intervals.
